(19) United States
(12) Patent Application Publication
(10) Pub. No.: US 2015/0265165 A1
(43) Pub. Date: Sep. 24, 2015

(54) System and Method For Non-Contrast Magnetic Resonance Imaging of Pulmonary Blood Flow

(71) Applicants: Iga Muradyan, Boston, MA (US); Samuel Patz, Chestnut Hill, MA (US); Ravi Teja Seethamraju, Malden, MA (US); Ritu Randhawa Gill, Lexington, MA (US)

(72) Inventors: Iga Muradyan, Boston, MA (US); Samuel Patz, Chestnut Hill, MA (US); Ravi Teja Seethamraju, Malden, MA (US); Ritu Randhawa Gill, Lexington, MA (US)

(21) Appl. No.: 14/664,583

(22) Filed: Mar. 20, 2015

Related U.S. Application Data
(60) Provisional application No. 61/968,858, filed on Mar. 21, 2014.

(51) Int. Cl. A61B 5/026; A61B 5/08; A61B 5/00

(52) U.S. Cl. CPC A61B 5/0263; A61B 5/004; A61B 5/743; A61B 5/08; A61B 2576/02

(57) ABSTRACT

A system and method for non-contrast imaging of pulmonary blood flow in a subject are described. In some aspects, the method includes acquiring, using a magnetic resonance imaging (“MRI”) system, image data from at least the subject's lungs during which little to no respiratory motion occurs in the subject, such as during a breath-hold. The method also includes assembling the image data into a plurality of time-series datasets representing temporal variations of magnetic resonance signals in a region of interest that contains all or part of the subject's lungs. The method further includes computing a statistical blood flow metric for each voxel in the region of interest, using respective time-series datasets, and generating at least one image representative of pulmonary blood flow in the subject using the computed statistical blood flow metrics.

### CROSS REFERENCE

This application is based on, claims priority to, and incorporates herein by reference in their entirety U.S. Ser. No. 61/968,858 filed Mar. 21, 2014 and entitled “NON-CONTRAST BREATH HELD MR PERFUSION OF THE LUNGS FROM TIME SERIES ANALYSIS.”

### BACKGROUND

The present disclosure relates generally to systems and methods for medical imaging and, in particular, to systems and methods for assessing pulmonary blood flow using magnetic resonance imaging (“MRI”).

Imaging of pulmonary blood flow using magnetic resonance has long faced challenges due to low signal-to-noise ratios (“SNR”). This is because MRI detects signals associated with protons in water molecules found in the body. However, even at rest, the majority of lung volume is occupied by air, which has a low proton density and results in low magnetic resonance signals. In addition, lung imaging is also exacerbated by the presence of air-tissue interfaces in the lung, which create magnetic susceptibility effects that deteriorate magnetic resonance signals.

One approach to deal with the inherently low magnetic resonance signals in the lungs has included the use of intravenous contrast agents to improve SNR. However, contrast agents, typically containing gadolinium, are limited in the amount and time over which they can be administered, as well as the time it takes for the contrast bolus to clear the blood, restricting the number of measurements and study repeatability. In addition, although blood volume may be readily obtained from measured tissue concentration curves, determining blood flow and transit time present additional difficulties and may be subject to appreciable errors.

Other approaches for imaging the lungs have included use of hyperpolarized gas, oxygen enhanced MRI techniques, ultrashort echo-time (“UTE”) or zero echo-time (“ZTE”) techniques, and Fourier decomposition. Fourier decomposition imaging of the lungs acquires a continuous set of magnetic resonance images during free breathing, typically using a balanced steady state free precession (“bSSFP”) pulse sequence. Because these images correspond to different time points in the breathing cycle, they are subjected to a spatial registration procedure in which the position and size of each image voxel is registered to a corresponding voxel in a reference image. The result of this operation is a time-series for each voxel in the spatially registered image. The time series from each spatially registered voxel is then Fourier transformed to obtain frequency spectra that depict frequency components modulating voxel intensities. For lung imaging, peaks in the respective spectra are representative of breathing and heart rate frequencies, which may then be used to identify the amplitude of regional proton density changes related to ventilation and blood flow, respectively.

Imaging the lungs using a bSSFP pulse sequence, however, produces known dielectric and off-resonance effect artifacts, particularly at high magnetic fields and large fields of view. Also, depending on the flip angle used and the repetition rate of the excitation pulses, the number of repeated measurements may be limited due to specific absorption rate (“SAR”) limitations. Most importantly, however, no matter what imaging pulse sequence is used, image registration produces appreciable errors since, in general, spatial registration algorithms only work well in regions of high SNR. In the case of the lungs, high SNR areas occur at the lung boundaries, heart tissues, and relatively large blood vessels. Other areas of the lung contain mostly air, which does not provide an appreciable signal. Thus, displacements during free breathing of small blood vessels that are part of the parenchymal tethered network, especially in the periphery of the lungs, are very difficult to register. Furthermore, the above Fourier decomposition approach for generating blood flow maps utilizes the spectral peak at the heart rate frequency only and ignores signal modulations produced at other frequencies by the heart.

Other approaches for imaging pulmonary blood flow have included arterial spin labeling (“ASL”), or blood tagging techniques. Specifically, ASL involves magnetically tagging arterial blood before entering a target tissue, and examining the amount of labeling measured in an image slice compared to a control obtained prior to labeling. However, ASL is rather difficult to implement in the thoracic region. Among other reasons, this is because the SNR of ASL is inherently low, given that signal from labeled inflowing blood is only about 1% of the full tissue signal.

Therefore, given the above, there is a need for systems and methods for improved imaging of pulmonary blood flow using MRI.

### SUMMARY

The present invention overcomes the aforementioned drawbacks by providing a system and method directed to magnetic resonance imaging of the lungs. Specifically, the present disclosure describes an approach for assessing pulmonary blood flow without need for contrast agent enhancement or blood tagging imaging. Also, the present approach overcomes signal contamination due to motion by performing an image acquisition during a period during which little to no respiratory motion occurs in a subject. In some aspects, a statistical metric for assessing blood flow is utilized, which captures information related to signal intensity modulations from all frequencies associated with the cardiac cycle. This approach produces appreciably enhanced blood flow maps when compared to observing the modulation solely at the heart rate.

In accordance with one aspect of the disclosure, a method for non-contrast imaging of pulmonary blood flow in a subject is provided. The method includes acquiring, using the MRI system, image data from at least the subject's lungs during a period when substantially no respiratory motion occurs in the subject, and assembling the image data into a plurality of time-series datasets representing temporal variations of magnetic resonance (“MR”) signals in a region of interest containing at least a part of the subject's lungs. The method also includes computing a statistical blood flow metric for each voxel in the region of interest using respective time-series datasets, and generating a report indicative of pulmonary blood flow in the subject using the computed statistical blood flow metrics.

In accordance with another aspect of the disclosure, a magnetic resonance imaging (“MRI”) system for non-contrast imaging of pulmonary blood flow in a subject is provided. The system includes a magnet system configured to generate a polarizing magnetic field about at least a portion of a subject arranged in the MRI system, a plurality of gradient coils configured to establish at least one magnetic gradient field to the polarizing magnetic field, and a radio frequency (“RF”) system configured to apply an RF field to the subject and to receive magnetic resonance (“MR”) signals therefrom. The system also includes a computer system programmed to direct the RF system and plurality of gradient coils to acquire image data from at least the subject's lungs during a period when substantially no respiratory motion occurs in the subject, and assemble the image data into a plurality of time-series datasets representing temporal variations of magnetic resonance (“MR”) signals in a region of interest containing at least a part of the subject's lungs. The computer system is also programmed to compute a statistical blood flow metric for each voxel in the region of interest using respective time-series datasets, and generate a report indicative of pulmonary blood flow in the subject using the computed statistical blood flow metrics.

The foregoing and other advantages of the invention will appear from the following description.

### BRIEF DESCRIPTION OF THE DRAWINGS

FIG. 1 is a flowchart setting forth the steps of an example of a method for non-contrast imaging of pulmonary blood flow in a subject;

FIG. 2A shows an example time-series dataset from a region of interest in the anterior segmental pulmonary artery of a subject;

FIG. 2B shows the Fourier spectrum of the time-series dataset shown in FIG. 2A;

FIG. 3A is an example Fourier component map in accordance with aspects of the present disclosure.

FIG. 3B is an example mean intensity map obtained in accordance with aspects of the present disclosure.

FIG. 3C is an example standard deviation map obtained in accordance with aspects of the present disclosure.

FIG. 4 is a block diagram of an example of a magnetic resonance imaging (“MRI”) system.

### DETAILED DESCRIPTION

The present disclosure provides a system and method for assessing pulmonary blood flow using non-contrast magnetic resonance imaging (“MRI”). In general, blood flow measurements are performed either by utilizing contrast agent enhancement or blood tagging techniques. While side effects, such as nephrogenic systemic fibrosis (“NSF”), have necessitated a reduction in the use of gadolinium-based contrast agents, particularly for patients with kidney failure, blood tagging has often proven difficult to implement when imaging the thoracic region.

Therefore, a novel approach is provided for determining pulmonary blood flow. In some aspects, the disclosed method applies a data acquisition approach that allows for fewer artifacts in comparison with previous data acquisition techniques. In particular, data may be acquired during a period of time during which little to no respiratory motion occurs in a subject, such as during a period of breath holding, so that motion-induced artifacts are appreciably reduced or eliminated. In addition, in applying a Fourier decomposition approach to map pulmonary blood flow, statistical quantities, such as the mean and standard deviation of intensities obtained from assembled time-series datasets corresponding to voxels in an imaging slice, are utilized. As will be appreciated, the disclosed technique may find a variety of applications, including helping to evaluate thoracic diseases non-invasively, and without the need for the administration of a contrast agent. Although reference is made herein specifically with reference to assessing pulmonary blood flow it may be appreciated that the approach of the present disclosure may be applicable to other areas of the body as well.

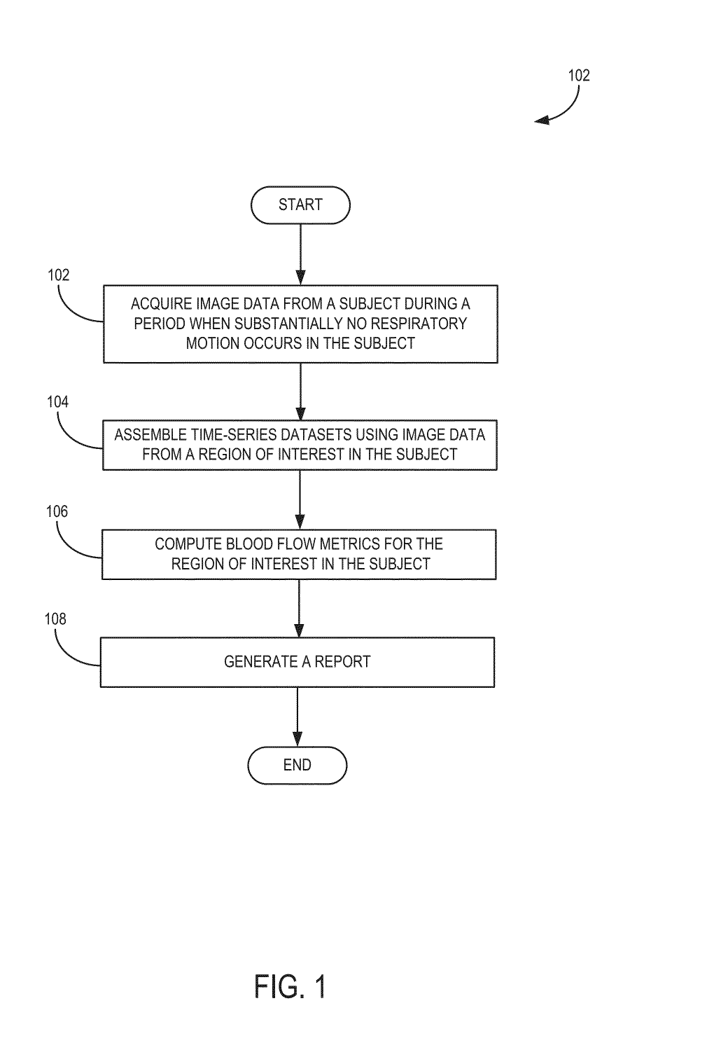

Turning to FIG. 1, steps of a process 100, in accordance with aspects of the present disclosure are shown. The process 100 may begin at process block 102, wherein 2-dimensional or 3-dimensional image data may be acquired from at least the subject's lungs using an MRI system. Previous Fourier decomposition techniques have aimed to quantify ventilation and blood flow in the lungs by acquiring free breathing time series data using a balanced steady state free precession (“bSSFP”) pulse sequence. Such free breathing techniques necessitate use of image registration algorithms, which are prone to appreciable error. Therefore, image data may be advantageously acquired at process block 102 during a period during which little to no respiratory motion occurs, such as during breath-hold, thereby reducing or eliminating artifacts induced by respiratory motion. In addition, bSSFP can only be used with a fair degree of success at magnetic fields of about 1.5 Tesla, due to artifacts associated with dielectric and off-resonance effects at higher fields. To overcome such shortcomings, an ultrafast gradient echo pulse sequence, such as a TurboFLASH pulse sequence, may be utilized at process block 102. It will be appreciated by one of ordinary skill that other pulse sequences affording sufficiently high frame rate may also be utilized at process block 102 to acquire image data.

Advantageously, by acquiring image data during breath holding, respiratory-induced artifacts can be virtually eliminated without need for performing image registration. By way of example, the image data can be acquired at process block 102 in a single breath hold over a period of, say, about 30 seconds. However, it may be appreciated that image acquisition may depend upon the duration of time that a subject can abstain from breathing. For instance, in cases where a subject may not be capable of holding their breath for a sufficiently long period of time, image data may be acquired using more than one breathing cycle. In such cases, computed statistical blood flow maps generated from separate breath-holds may be combined, for example, using a simple spatial registration. This is in contrast to the complex registration algorithms applied in free-breathing techniques, which utilize image data acquired in the same phase of the breathing cycle, typically the end of inspiration or expiration.

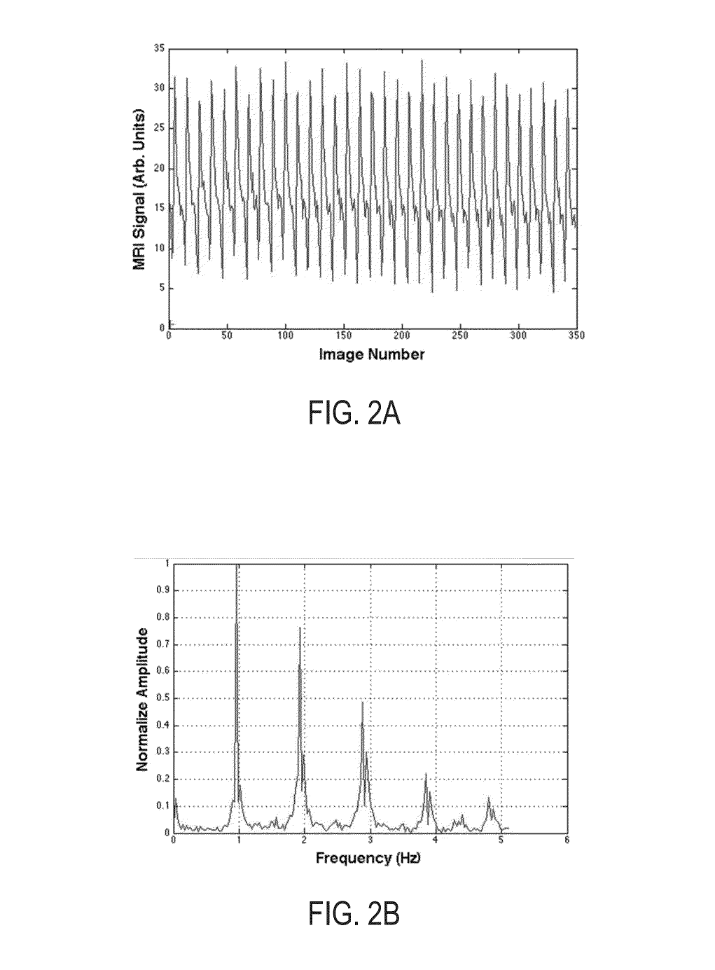

At process block 104 time-series datasets may be assembled using images reconstructed using the acquired image data. Specifically, each time-series dataset represents temporal variations of magnetic resonance (“MR”) signals for a pixel or voxel in a region of interest within, or about, a subject's lungs. By way of example, FIG. 2A shows an example time-series dataset from a region of interest that is roughly 10 millimeters in diameter and located in the anterior segmental pulmonary artery of a subject.

Then, various metrics indicative of blood flow are computed at process block 106 using the assembled time-series datasets. In some aspects, similar to previous Fourier decomposition approaches, spectral distributions indicating spectral components of the time-series datasets may be obtained by applying a Fourier analysis. By way of example, FIG. 2B shows the spectral distribution for the example time-series dataset shown in FIG. 2A. The spectral distribution contains several peaks, some of which are related to density changes occurring at or near the frequency of the subject's heart rate, as well as higher frequency harmonics. Using this spectral information from the determined spectral distribution, a metric indicative of blood flow can be computed for each pixel or voxel. In some aspects, a blood flow metric may be computed by integrating the spectral distribution around a frequency interval centered about the heart rate frequency. It may be appreciated, however, that other metrics may be computed using spectral information associated with peaks present in the distribution.

In accordance with other aspects, blood flow metrics can be obtained at process block 106 by computing statistical quantities, such as the mean and/or the standard deviation of intensities for the assembled time-series datasets. As described, such statistical blood flow metrics need not be limited to obtaining information from a single frequency, such as the heart rate frequency or higher harmonics, but instead may advantageously capture signal intensity modulations from multiple frequencies, thereby producing appreciably enhanced blood flow maps.

Then, at process block 108, a report indicative of pulmonary blood flow in the subject may be generated using the computed blood flow metrics, and other information, determined at process block 106. In some aspects, the report may be in the form of blood flow maps, such as Fourier component maps, mean intensity maps, standard deviation maps, and so on, or combinations thereof. In other aspects, the report may include information related to an influx of blood into an imaging slice, or an in-plane motion of a blood vessel, or a change in blood volume for an imaging slice, or combinations thereof. In addition, the report may include information related to a medical condition of the subject, such as a thoracic disease.

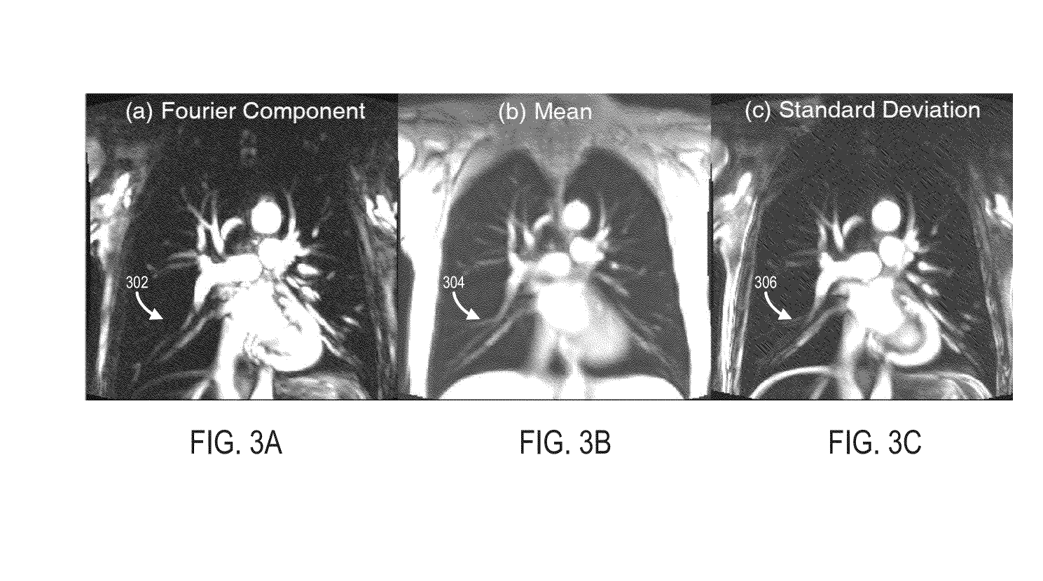

By way of a non-limiting example, coronal 2D TurboFLASH breath-held scans were performed on 4 healthy subjects at 3 Tesla. Acquisition parameters included TE/TR=1.1/98 ms, FOV=305 mm, data matrix=96×72 (interpolated to 256×256), turbo factor=116, α=20 degrees and 4 mm slices. It may be appreciated, however, that other imaging parameters may be utilized. FIG. 3 shows an example of blood flow maps obtained from the acquired image data in accordance with the method of the present disclosure. Specifically, FIG. 3A shows an example Fourier component map, FIG. 3B shows a mean intensity map, and FIG. 3C shows a standard deviation map. When comparing these maps, it can be seen that the Fourier component map (FIG. 3A) shows lower signal-to-noise ratio, and poorer vasculature conspicuity than the other maps. For example, vasculature visible on the mean intensity (FIG. 3B) and standard deviation maps (FIG. 3C), indicated by arrows 304 and 306, respectively, is not visible on the Fourier component map (FIG. 3A), as indicated by arrow 302.

The vascular contrast for the mean intensity map is primarily due to the difference in spin density between vasculature, and surrounding parenchyma. The Fourier component map and standard deviation maps, however, are additionally sensitive to modulations in the signal intensity either at the heart rate or all frequencies, respectively. Sources of signal intensity modulation include the influx of fresh blood into the slice for each heart beat, heart beat related in-plane motion of the blood vessels, and changes in blood volume. Blood vessels with primarily in-plane flow will have a reduced sensitivity to fresh blood inflow. The blood flow visible in FIG. 3 near arrow 306, and not visible near arrow 302, implies that there are signal intensity modulations at frequencies other than the heart rate.

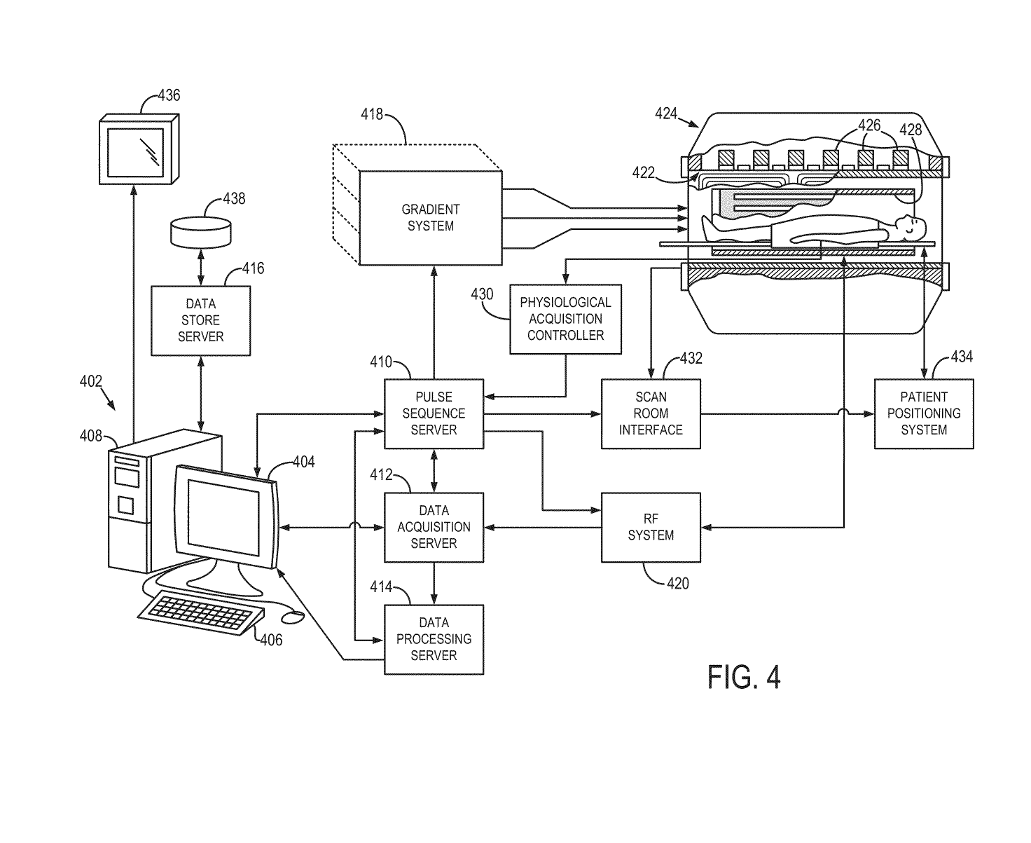

Referring now to FIG. 4, an example of a magnetic resonance imaging (“MRI”) system is illustrated. The MRI system includes a workstation 402 having a display 404 and a keyboard 406. The workstation 402 includes a processor 408, such as a commercially available programmable machine running a commercially available operating system. The workstation 402 provides the operator interface that enables scan prescriptions to be entered into the MRI system. The workstation 402 is coupled to four servers: a pulse sequence server 410; a data acquisition server 412; a data processing server 414, and a data store server 416. The workstation 402 and each server 410, 412, 414 and 416 are connected to communicate with each other.

The pulse sequence server 410 functions in response to instructions downloaded from the workstation 402 to operate a gradient system 418 and a radio frequency (“RF”) system 420. Gradient waveforms necessary to perform the prescribed scan are produced and applied to the gradient system 418, which excites gradient coils in a gradient coil assembly 422 to produce the magnetic field gradients Gₓ, G_{y}, and G_{z} used for position encoding MR signals. The gradient coil assembly 422 forms a part of a magnet assembly 424 that includes a polarizing magnet 426 and a whole-body RF coil 428.

RF excitation waveforms are applied to the RF coil 428, or a separate local coil (not shown in FIG. 4), by the RF system 420 to perform the prescribed magnetic resonance pulse sequence. Responsive MR signals detected by the RF coil 428, or a separate local coil (not shown in FIG. 4), are received by the RF system 420, amplified, demodulated, filtered, and digitized under direction of commands produced by the pulse sequence server 410. The RF system 420 includes an RF transmitter for producing a wide variety of RF pulses used in MR pulse sequences. The RF transmitter is responsive to the scan prescription and direction from the pulse sequence server 410 to produce RF pulses of the desired frequency, phase, and pulse amplitude waveform. The generated RF pulses may be applied to the whole body RF coil 428 or to one or more local coils or coil arrays (not shown in FIG. 4).

The RF system 420 also includes one or more RF receiver channels. Each RF receiver channel includes an RF amplifier that amplifies the MR signal received by the coil 428 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I₂+Q₂} (4);

and the phase of the received MR signal may also be determined:

$$φ = {tan}^{- 1} ( \frac{Q}{I} ) . \qquad ( 5 )$$

The pulse sequence server 410 also optionally receives patient data from a physiological acquisition controller 430. The physiological acquisition controller 430 receives signals from a number of different sensors connected to the patient, such as electrocardiograph (“ECG”) signals from electrodes, or respiratory signals from a bellows or other respiratory monitoring device. Such signals are typically used by the pulse sequence server 410 to synchronize, or “gate,” the performance of the scan with the subject's heart beat or respiration.

The pulse sequence server 410 also connects to a scan room interface circuit 432 that receives signals from various sensors associated with the condition of the patient and the magnet system. It is also through the scan room interface circuit 432 that a patient positioning system 434 receives commands to move the patient to desired positions during the scan.

The digitized MR signal samples produced by the RF system 420 are received by the data acquisition server 412. The data acquisition server 412 operates in response to instructions downloaded from the workstation 402 to receive the real-time MR data and provide buffer storage, such that no data is lost by data overrun. In some scans, the data acquisition server 412 does little more than pass the acquired MR data to the data processor server 414. However, in scans that require information derived from acquired MR data to control the further performance of the scan, the data acquisition server 412 is programmed to produce such information and convey it to the pulse sequence server 410. For example, during prescans, MR data is acquired and used to calibrate the pulse sequence performed by the pulse sequence server 410. Also, navigator signals may be acquired during a scan and used to adjust the operating parameters of the RF system 420 or the gradient system 418, or to control the view order in which k-space is sampled.

The data processing server 414 receives MR data from the data acquisition server 412 and processes it in accordance with instructions downloaded from the workstation 402. Such processing may include, for example: Fourier transformation of raw k-space MR data to produce two or three-dimensional images; the application of filters to a reconstructed image; the generation of functional MR images; and the calculation of motion or flow images.

Images reconstructed by the data processing server 414 are conveyed back to the workstation 402 where they are stored. Real-time images are stored in a data base memory cache (not shown in FIG. 4), from which they may be output to operator display 412 or a display 436 that is located near the magnet assembly 424 for use by attending physicians. Batch mode images or selected real time images are stored in a host database on disc storage 438. When such images have been reconstructed and transferred to storage, the data processing server 414 notifies the data store server 416 on the workstation 402. The workstation 402 may be used by an operator to archive the images, produce films, or send the images via a network to other facilities.

Features suitable for such combinations and sub-combinations would be readily apparent to persons skilled in the art upon review of the present application as a whole. The subject matter described herein and in the recited claims intends to cover and embrace all suitable changes in technology.

1. A method for non-contrast imaging of pulmonary blood flow in a subject using a magnetic resonance imaging system (“MRI”) system, the method comprising:
a) acquiring, using the MRI system, image data from at least the subject's lungs during a period when substantially no respiratory motion occurs in the subject;
b) assembling the image data into a plurality of time-series datasets representing temporal variations of magnetic resonance (“MR”) signals in a region of interest containing at least a part of the subject's lungs;
c) computing a statistical blood flow metric for each voxel in the region of interest using respective time-series datasets; and
d) generating a report indicative of pulmonary blood flow in the subject using the statistical blood flow metrics computed at step c).

2. The method of claim 1, wherein the statistical blood flow metric is a mean intensity of MR signals in a time-series dataset.

3. The method of claim 2, further comprising generating a mean intensity map using the computed statistical blood flow metrics, wherein the mean intensity map indicates at least a contrast between a vasculature and a lung parenchyma.

4. The method of claim 1, wherein the statistical blood flow metric is a standard deviation of MR signals in a time-series dataset.

5. The method of claim 4, further comprising generating a standard deviation map using the computed statistical blood flow metric, wherein the standard deviation map indicates signal intensity modulations at multiple frequencies.

6. The method of claim 1, wherein the report includes at least one image representative of pulmonary blood flow in the subject.

7. The method of claim 1, wherein the report includes information related to at least one of an influx of blood into an imaging slice for each heart beat, an in-plane motion of a blood vessel, or a change in blood volume for the imaging slice.

8. The method of claim 1, wherein step a) includes directing the MRI system to apply an ultrafast gradient echo pulse sequence to acquire the image data.

9. The method of claim 8, wherein step d) includes generating a statistical blood flow map using the computed statistical blood flow metric, the statistical blood flow map indicating a measure of pulmonary blood flow in the subject.

10. The method of claim 9, wherein the statistical blood flow metric is a mean intensity of MR signals in a time-series dataset.

11. The method of claim 9, wherein the statistical blood flow metric is a standard deviation of MR signals in a time-series dataset.

12. A magnetic resonance imaging (“MRI”) system for non-contrast imaging of pulmonary blood flow in a subject, the system comprising:
a magnet system configured to generate a polarizing magnetic field about at least a portion of a subject arranged in the MRI system;
a plurality of gradient coils configured to establish at least one magnetic gradient field to the polarizing magnetic field;
a radio frequency (“RF”) system configured to apply an RF field to the subject and to receive magnetic resonance (“MR”) signals therefrom;
a computer system programmed to:
i) direct the RF system and plurality of gradient coils to acquire image data from at least the subject's lungs during a period when substantially no respiratory motion occurs in the subject;
ii) assemble the image data into a plurality of time-series datasets representing temporal variations of magnetic resonance (“MR”) signals in a region of interest containing at least a part of the subject's lungs;
iii) compute a statistical blood flow metric for each voxel in the region of interest using respective time-series datasets; and
iv) generate a report indicative of pulmonary blood flow in the subject using the statistical blood flow metrics computed at step iii).

13. The system of claim 12, wherein the statistical blood flow metric is a mean intensity of MR signals in a time-series dataset.

14. The system of claim 13, wherein the computer is further programmed to use the computed mean intensities to generate a mean intensity map indicating at least a contrast between a vasculature and a lung parenchyma.

15. The system of claim 12, wherein the statistical blood flow metric is a standard deviation of MR signals in a time-series dataset.

16. The system of claim 15, wherein the computer is further programmed to use the computed standard deviations to generate a standard deviation map indicative of signal intensity modulations at multiple frequencies.

17. The system of claim 12, wherein the report includes at least one image representative of pulmonary blood flow in the subject.

18. The system of claim 12, wherein the report includes information related to at least one of an influx of blood into an imaging slice for each heart beat, or an in-plane motion of a blood vessel, or a change in blood volume for the imaging slice.

19. The system of claim 12, wherein the computer is further programmed to direct the MRI system to apply an ultrafast gradient echo pulse sequence to acquire the image data for use in generating a statistical blood flow map.

20. The system of claim 19, wherein the statistical blood flow map is at least one of a mean intensity map or a standard deviation map.
